(19) United States
(12) Patent Application Publication
(10) Pub. No.: US 2004/0122345 A1
(43) Pub. Date: Jun. 24, 2004

(54) Body fluid aspiration and injection syringe

(76) Inventor: Hans Muller, Munchen (DE)

Correspondence Address: BIRDWELL, JANKE & DURANDO, PLC, Suite 1400, 1100 SW Sixth Avenue, Portland, OR, 97204, US

(21) Appl. No.: 10/649,555

(22) Filed: Aug. 26, 2003

Related U.S. Application Data
(63) Continuation-in-part of application No. 10/048,693, filed on Jan. 31, 2002, now abandoned.
Application No. 10/048,693 is a 371 of international application No. PCT/EP00/06892, filed on Jul. 19, 2000.

(30) Foreign Application Priority Data
Aug. 2, 1999 (DE) 199 36 294-7

(51) Int. Cl. A61M 37/00

(52) U.S. Cl. 604/6.14; 604/6.08

(57) ABSTRACT

The invention resides in a syringe which can be evacuated and which includes a piston rod structure that can be locked in predetermined positions. The syringe is particularly suitable for the treatment of body fluids outside the body particularly for the ultraviolet irradiation treatment of blood or HOT treatment of blood.

### CROSS-REFERENCE TO RELATED APPLICATIONS

This application is a continuation-in-part of U.S. patent application Ser. No. 10/048,693, filed Jan. 31, 2002, which is incorporated herein by reference.

### STATEMENT REGARDING FEDERALLY SPONSORED RESEARCH OR DEVELOPMENT

Not Applicable.

### INCORPORATION-BY-REFERENCE OF MATERIAL SUBMITTED ON A COMPACT DISC

Not Applicable.

### BACKGROUND OF THE INVENTION

1. Field of the Invention

The invention relates to a syringe consisting of a cylinder for receiving body fluids which narrows down, at the front end, to form an insertion tip and which, at its rear end, includes an outwardly extending holding flange as well as a piston mounted on a piston rod which includes a retaining plate. The piston is movable in the cylinder and includes a piston rod structure which is lockable at pre-selectable positions of the piston in the cylinder.

Such a syringe is used particularly as a containment for the treatment of body fluids outside the body, for example for a UV-irradiation treatment (UVT) or a hematogeneous oxidation therapy (HOT).

With the hematogeneous oxidation therapy, the blood extracted from the body of the patient is treated with simple or with activated oxygen, so-called Singulett-oxygen or other therapeutic gases. Below, for simplicity reasons, all the therapeutic gases will be called “oxygen”.

2. Description of Related Art

DE 13 30189 discloses the treatment of blood by UV irradiation wherein a conventional hand-operated syringe is used as a receiving container for the blood.

Instead of the conventional hand-operated syringes, DE-OS 39 32 109 provides, for the withdrawal of blood, a syringe with locking means on the piston rod structure, which is intended to prevent re-infusion of the blood. This locking means also permits the locking of the piston in predetermined axial positions. Locking means for the same purpose are also disclosed in EP-A-35 4824, FR 02536285 and DE Utility Model 7935103.

In order to facilitate the withdrawal of blood, rather than into syringes, the body liquids are generally drawn during removal into vacuum bottles by a vacuum provided in the bottles. The body fluid collected in these vacuum bottles can then also be treated with oxygen by connecting the bottle to an oxygen source by a coupling element and a communication hose. Subsequently, the body fluid treated in the vacuum bottle is reintroduced into the patient by means of well-known transfusion equipment.

An important disadvantage of such vacuum bottles is that the quality of the vacuum within the bottle cannot be tested from the outside. If the vacuum is insufficient, an insufficient amount of body fluid is withdrawn from the patient. Such an insufficient sample must then be discarded together with the bottle and the drawing of body fluid must be repeated with a new vacuum bottle. In addition to the material expenses, this represents an unjustifiable stress for the patient.

Another disadvantage of such vacuum bottles is their volume requiring a relatively large amount of space and the storage, transport and safe disposal costs. In addition, there is the danger of breakage if the vacuum bottles consist of glass.

DE Utility Model 94 21 606 discloses an alternative container for an apparatus for the extra-corporal treatment of blood. In that case, the blood is not treated with therapeutic gases in a vacuum bottle but in a flexible plastic bag. Such plastic bags are very space saving. But since they cannot maintain a vacuum, only gravity forces can be used to withdraw the blood. This increases the time required for withdrawing the blood and is therefore inconvenient for a patient.

U.S. Pat. No. 5,697,915 discloses multi-chambered syringes which are used for sequential inspection and/or the mixing of drugs and solutions. The sequential aspiration, mixing and injection syringe includes a cylinder which is sized to receive a piston assembly which comprises a piston rod structure and a vial at the distal end thereof. This vial comprises in a vial chamber a piston and a distal stopper with holes leading to the interior space of the cylinder. The drug to be mixed with diluent fluid is placed within the vial chamber. After the distal stopper has been tightly positioned at the distal end of the cylinder the drug is pushed out of the vial chamber by retracting the piston rod structure. At the same time a nascent mixing is formed intermediate the distal stopper and the vial chamber. With the further retracting of the piston rod structure diluent fluid passes through the holes at the distal stopper into the mixing chamber.

In a further embodiment of a multi-chambered syringe with a flexible diluent reservoir a check valve is provided which is opened by a relative negative pressure when the piston rod structure is retracted in order to collapse the flexible reservoir and to push diluent into the mixing chamber.

The syringes disclosed in U.S. Pat. No. 5,697,915 are not suitable for UV-irradiation treatment or a hematogeneous oxidation therapy of body fluids or blood.

U.S. Pat. Nos. 5,533,970 and 6,164,348 disclose locking means for arranging the piston rod structure at certain positions in the cylinder of a syringe. They are used for totally different utilization.

### BRIEF SUMMARY OF THE INVENTION

It is the object of the present invention to provide a container for receiving body fluids, which does not have the disadvantages of the blood containers known from the state of the art and which can replace a vacuum bottle. For cost reasons and for ensuring universal applicability, the container should have small equipment expenses and should be widely useable.

The object is solved based on the initially described syringe, which comprises:

a cylinder having a converging distal end forming a first closeable inserting tip adapted for receiving body fluids aspirated through said first inserting tip;

a piston rod structure which is essentially cross-shaped extending in this form essentially over the full axial length of said cylinder, said piston rod structure having a longitudinal bore extending through the center portion of said piston rod structure from, a second inserting tip adapted be closed at the proximal end thereof, to a check valve at the distal end thereof;

said piston rod structure comprising a locking device for locking said piston rod structure in said cylinder, said locking device being arranged at positions in said piston rod structure that correspond to various syringe fill volumes;

a cap seal structure permanently fixed to said distal end of said piston rod structure, said cap seal structure being provided with holes leading from the interior of said cylinder to an interior space defined between said cap seal structure and said check valve at the distal end of said longitudinal bore; and

said syringe being utilized for creating a vacuum in said cylinder such that, after said first and second inserting tips at the proximal and the distal ends being closed, said piston rod structure is pulled out of said cylinder up to a locking position and locked therein in order to use said vacuum for aspiration of blood after said first inserting tip at the distal end being opened.

Further developments of the invention are subject of dependent claims.

The various embodiments of the syringe according to the invention provide an advantageous and relatively inexpensive substitute for vacuum bottles consisting of glass. The syringe can be used for all types of uses of vacuum bottles. Since the vacuum is generated only when needed by pulling the piston out of the syringe cylinder, the problems occurring with the loss of vacuum of the vacuum bottles during storage are eliminated.

With the lockability of the insertion syringe and the provision of openings in the cover seal of the piston head which are in communication with the longitudinal bore by way of a blocking of return-flow the syringe is very suitable for the hematogeneous oxidation therapy.

The rear end of the longitudinal bore can be closed during the drawing of blood by a one-way valve. The longitudinal bore includes behind the cover seal the blocking for return-flow e.g., a check valve, preferably a double flap valve in order to prevent blood from entering the longitudinal bore.

### BRIEF DESCRIPTION OF THE DRAWINGS

The advantages and features of the invention will become apparent from the description of the embodiments in connection with the claims and the drawings. It is shown in:

FIG. 1 a longitudinal cross-sectional view of the syringe according to the invention;

FIG. 2 the syringe according to FIG. 1 with the piston pulled out of the syringe cylinder;

FIG. 3 a cross-section taken along line III-III of FIG. 2;

FIG. 4 a longitudinal cross-sectional view of another embodiment of the invention;

FIG. 5 a syringe according to FIG. 4 with the piston pulled out of the syringe cylinder and the piston rod structure locked;

FIG. 6 a longitudinal cross-sectional view taken along line VI-VI of FIG. 5;

FIG. 7 another embodiment of the syringe with locking members disposed on the piston rod structure;

FIG. 8 a cross-sectional view taken along line IX-IX of FIG. 7.

FIG. 9 a partial longitudinal cross-sectional view of the syringe according to FIG. 7 with locked piston rod structure;

FIG. 10 a use of the syringe according to the invention for the UV treatment (UVT) or, respectively, Hematogeneous Oxidation Therapy (HOT).

### DETAILED DESCRIPTION OF THE INVENTION

In the following description of the figures the same parts are designated by the same reference numerals.

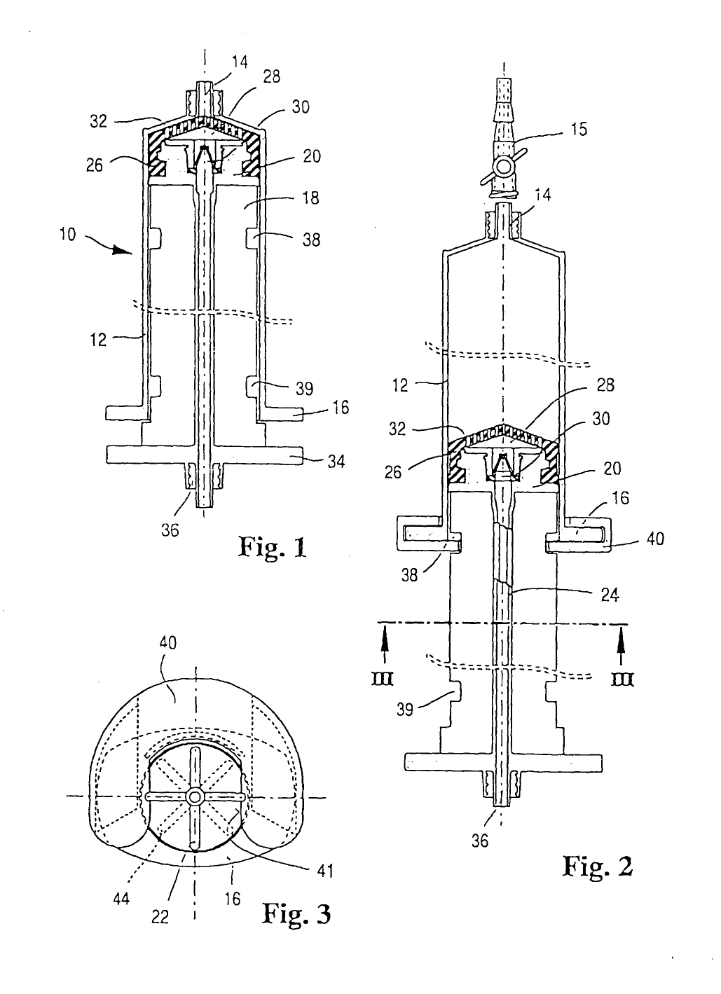

The syringe 10 shown in FIGS. 1 to 3 consists of a syringe cylinder 12 for receiving a body fluid, which cylinder narrows down at its front end to an insertion tip 14 and which has at its rear end an outwardly extending retaining flange 16. Movably, in the interior of the syringe cylinder 12, there is disposed a piston head 20, which is provided with a piston rod structure 18. The piston rod structure 18 consists of cross-webs 22 with a center portion through which a longitudinal bore 24 extends. A cap seal structure 26 is disposed on the piston head 20, which is adapted to the narrowed front end of the piston head. Between the cap seal structure 26 and the piston head, there is a space 28, which is in communication with the front end of the longitudinal bore 24, which is provided with a double flap check valve 30. Instead of a double flap check valve also a bacteria filter can be used which provides the same results. The cap seal structure 26 includes annually arranged passages 32 which lead to the space 28 and whose diameters preferably become increasingly smaller from the outer annulus toward the center of the cap seal structure 26. At their rearward end, the cross-webs 22 are provided with a retaining plate 34 through which the longitudinal bore 24 extends and which ends with an insertion tip 36. The front as well as the rear insertion tips 14 and 36, respectively, are preferably Luer-type connections into which a one-way valve can be inserted.

The cross-web-like piston rod 18 includes at its outer edges recesses 38, 39, which are arranged at predetermined locations. These locations correspond to predetermined syringe volumes. Preferably, the recesses 38, 39 are arranged at locations corresponding to syringe volumes of 10 ml and 60 ml and, for large volume syringes, of 120 ml.

For an embodiment of the syringe designed specifically for HOT treatment, the expandable volume in the syringe cylinder 12 is longer than the piston rod structure 18 in order to obtain additional free volume for the foaming (mixing) step.

For the use of the syringe as a vacuum syringe, a valve in the form of a one way valve 15 is placed onto the front and the rear insertion tips 14 and 36. Furthermore, there is provided a locking disc 40, which includes a U-shaped inner cutout 41 as shown in FIGS. 2 and 3 so that it can be moved past the retaining flange 16 and be received in the cutouts 38, 39, whereby the pulled out piston rod structure is locked in the pulled out position. If during pulling out of the piston the one-way valves disposed at the front and the rear insertion tips are closed, a low pressure or vacuum is generated in the syringe cylinder, which can be maintained by the locking of the piston rod-structure in a particular position by way of the locking disc.

For generating the vacuum with the syringe inlet closed the cross-web structure 22 of the piston rod is rotated to the angular position 44 as shown in dashed lines in FIG. 3 and is then pulled outwardly from the cylinder. As soon as the cutouts 38, 39 are disposed in the plane of the locking disc 40 disposed on the retaining flange 16 the piston is rotated to the position of the cross-web structure 22 as indicated in FIG. 3 by full lines, whereby the cutouts 38, or respectively, 39, are locked by the locking disc 40 and the vacuum is maintained.

The syringe evacuated in this manner can then be used like a vacuum bottle during the conventional treatment of body fluids, that is the syringe described herein can be used as a full substitute for a conventional vacuum bottle.

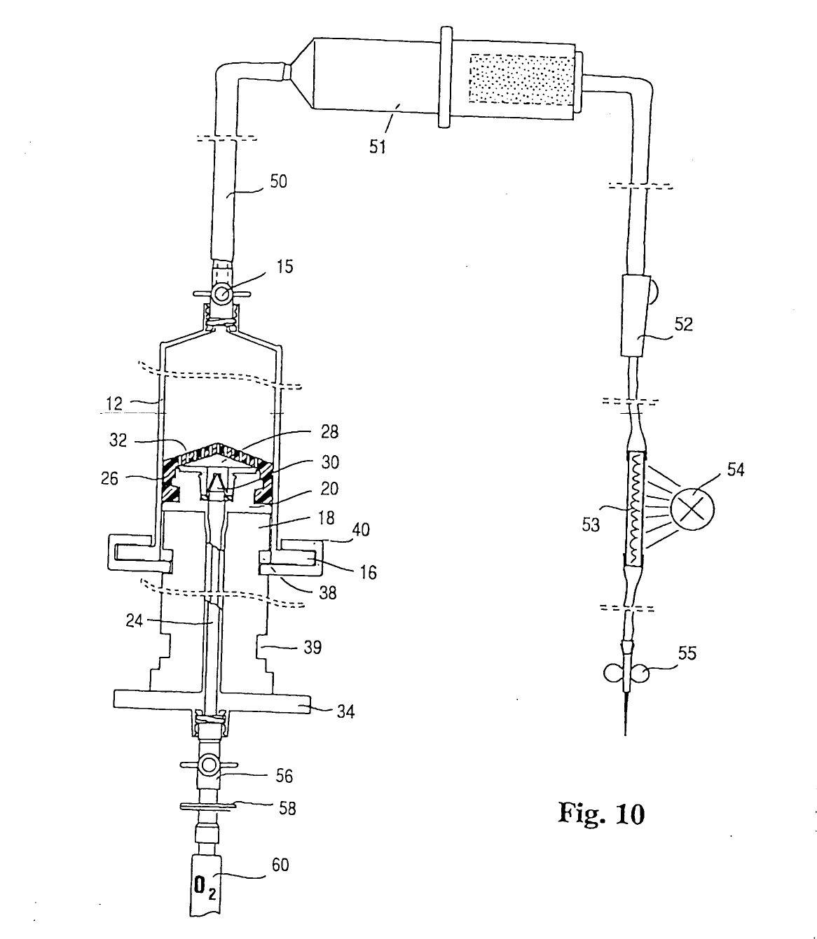

FIG. 10 shows a transfusion set for a UVT and/or HOT treatment. For such a treatment, blood is first withdrawn from a patient with the syringe according to the invention. For this purpose, preferably the one-way valve 15 is first mounted onto the Luer connection of the insertion tip 14 and is attached at the rear end of the longitudinal bore 24. With the valve 15 closed a vacuum is generated in the syringe cylinder 12 by manually pulling the piston out of the syringe cylinder 12. While the piston is pulled out with the cross-web structure 22 in the position 44, the piston may be locked by rotating the piston rod structure 18 by about 45° into a position in which the cutouts 39 or respectively, 38 of the piston rod structure engage the locking disc 40 disposed on the retaining flange 16. With the piston rod being locked a return of the piston by the vacuum forces on the syringe cylinder is prevented. As a result of this measure, the syringe can be handled easily and without efforts although a vacuum exists within the syringe as a result of the piston being in a pulled out position.

After a relative small vacuum has been generated in the syringe by the engagement of the locking disc 40 in the cutouts 39, a cannula, which is not shown in the drawings is placed onto the one-way valve 37 and for example sodium citrate is drawn into the syringe with the one-way valve opened because of the vacuum existing in the syringe.

For the withdrawal of blood from a patient with the one-way valve again closed the cannula is preferably replaced by a conventional transfusion set comprising a hose 50, a blood filter 51, a roller clamp 52 and, with UVT treatment, a cuvette 53 as well as a wing cannula 55. After further extraction of the piston and locking of the piston rod structure 18 in the cutouts 38 a vein of the patient is punctured by the wing cannula. By then opening the one-way valve 15, the blood is sucked into the syringe by the vacuum present in the syringe.

When a sufficient amount of blood has been drawn from the patient the locking mechanism is released in order to return sodium citrate-containing blood back into the patient through the wing cannula. As soon as thereafter the one-way valve 15 is closed a bacteria filter 58 is mounted on the one-way valve 56 on the rear insertion tip 36 for the HOT treatment and an oxygen source 60 is connected thereto. When the one-way valve 56 is then opened, oxygen can flow through the longitudinal bore 34, the double flap valve 30 and the holes 32 in the cover seal 26 into the interior of the syringe. The gas entering under pressure is pressed through the holes 32 and causes foaming of the blood. Subsequently, the oxygen is mixed with the blood by shaking while the one-way valve 56 is maintained closed until the blood bubbles have collapsed. The oxygen treatment is repeated until the color of the blood has changed its color to light red.

The syringe with the HOT-treated blood is then returned to the patient in a conventional manner by way of the transfusion set connected to the one-way valve 15. At the same time, the oxygen-enriched blood can be conducted through cuvette 53 for UV irradiation in an irradiation apparatus, which includes a UV radiation source 54. During irradiation, the flow speed of the blood is controlled preferably by a roller clamp 52.

After completed treatment of the blood, the blood is reinfused into the patient by way of the wing cannula 55.

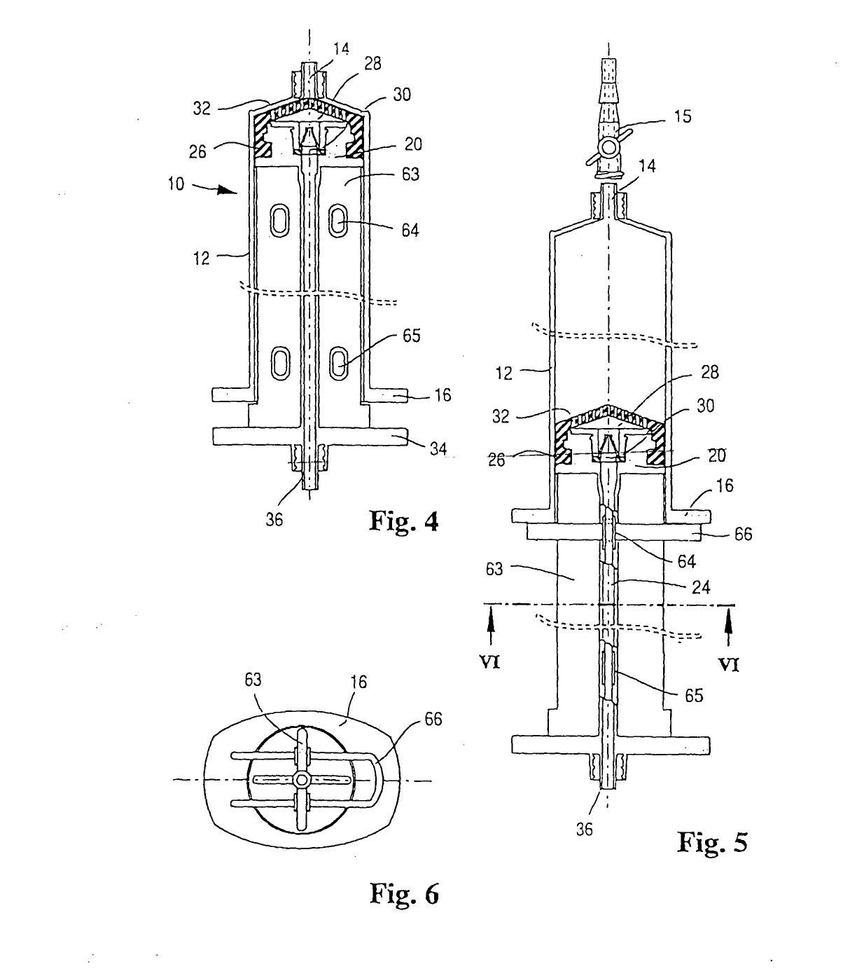

FIGS. 4 to 6 show another embodiment of the invention wherein the locking mechanism for the locking of the piston rod structure 63 with the retaining flange 16 is different. For locking the piston rod structure 63, the cross-web structures 22 include cutouts or, respectively, openings 64, 65 at the predetermined locations and a U-shaped locking pin 66 can be moved through the openings 64, 65 in order to lock the piston rod structure 63 with respect to the retaining flange 16 as shown in FIGS. 5 and 6.

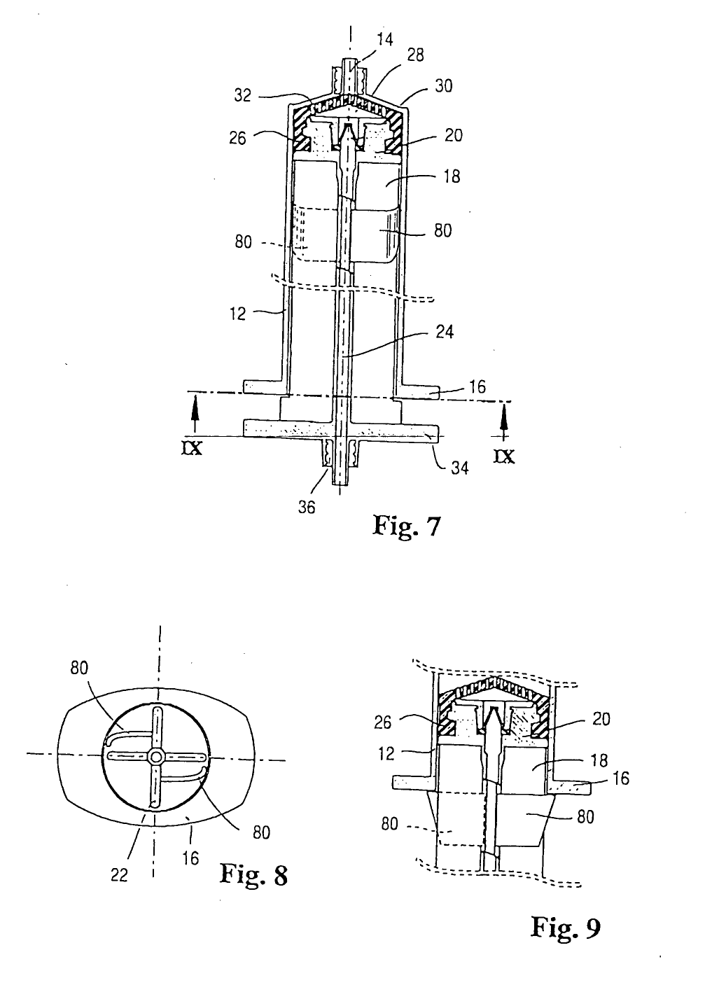

Finally, a particularly advantageous locking mechanism for the piston rod structure 18 with the retaining flange 16 is shown in FIGS. 7 to 9. In this case, preferably two cross-webs 22 of the piston rod structure include flexible locking arms 80, which extend parallel to a cross-web and in spaced relationship therefrom. They are at least as long as the cross-webs preferably somewhat longer at least at their front edge facing the piston head so that, after pull-out they can form a firm lock with the retaining flange according to FIG. 9.

During insertion of the piston rod structure into the syringe cylinder the locking arms 80 can easily flex backwardly as shown in FIG. 8, so that they abut the inner surface of the cylinder. Upon retraction of the piston rod structure, the locking arms spring back and provide for a safe locking of the piston rod structure. Several additional locking arms may be provided at the piston rod structure in order to provide additional locking positions.

1. A body fluid aspiration and injection syringe for UV-irradiation treatment or hematogeneous oxidation therapy of body fluids outside the body, said syringe comprising:
a) a cylinder having a converging distal end forming a first closeable inserting tip adapted for receiving body fluids aspirated through said first inserting tip;
b) a piston rod structure which is essentially cross-shaped extending in this form essentially over the full axial length of said cylinder, said piston rod structure having a longitudinal bore extending through the center portion of said piston rod structure from, a second inserting tip adapted to be closed at the proximal end thereof, to a check valve at the distal end thereof;
c) said piston rod structure comprising a locking device for locking said piston rod structure in said cylinder, said locking device being arranged at positions in said piston rod structure that correspond to various syringe fill volumes;
d) a cap seal structure permanently fixed to said distal end of said piston rod structure, said cap seal structure being provided with holes leading from the interior of said cylinder to an interior space defined between said cap seal structure and said check valve at the distal end of said longitudinal bore;
e) said syringe being utilized for creating a vacuum in said cylinder such that, after said first and second inserting tips at the proximal and the distal ends being closed, said piston rod structure is pulled out of said cylinder up to a locking position and locked therein in order to use said vacuum for aspiration of blood after said first inserting tip at the distal end being opened.

2. The body fluid aspiration and injection syringe of claim 1, further comprising a locking disk for placement on a retaining flange of said cylinder, said locking disc having a U-shaped inner recess for receiving said retaining flange and legs that fit into corresponding recesses in said piston rod structure such that rotating said piston rod structure causes said recesses of said piston rod structure to engage said legs and to lock said piston rod structure in a locking position.

3. The body fluid aspiration and injection syringe of claim 1, wherein said holes in said cap seal structure are largest at a distance closest to walls of said cylinder and decrease in size toward the center of the cylinder.

4. The body fluid aspiration and injection syringe of claim 2, wherein said recesses are arranged at locations in said piston rod structure that correspond to syringe fill volumes from about 1 ml to about 120 ml.

5. The body fluid aspiration and injection syringe of claim 1, wherein said distal end of said longitudinal bore is adapted to be connected to an oxygen source such that oxygen is supplied to the interior of said syringe by passing through said check valve and said holes in said cap seal structure in order to introduce oxygen into blood by foaming oxygen therein.

6. The body fluid aspiration and injection syringe of claim 1, wherein said piston rod structure includes at least one cut-out in opposite web areas of said piston rod structure, wherein an insertion pin is insertable into said at least one cut-out when said piston rod structure is pulled out of the cylinder such that said insertion pin engages the retaining flange and thereby locks said piston rod structure in its pulled-out position.

7. The body fluid aspiration and injection syringe of claim 6, wherein the at least one cut-out is arranged at locations in said piston rod structure that correspond to syringe fill volumes from about 1 ml to about 120 ml.

8. The body fluid aspiration and injection syringe of claim 1, wherein said essentially cross-shaped piston rod structure includes at least one web having at least one corresponding short web-like elastic locking arm extending parallel to an adjacent web, said at least one elastic locking arm extending outwardly and having at least part of an outer edge extending to an outer plane of said at least one web such that during insertion of the piston rod structure, said at least one locking arm is movable into the interior of the syringe by sideward flexing and that said at least one locking arm springs back out to extend over the retaining flange when the piston rod structure is moved out of said syringe.

9. The body fluid aspiration and injection syringe of claim 8, wherein the insertion tip of the cylinder and said bore at said distal end of said piston rod structure are closed and said piston rod structure is pulled out of the cylinder up to said locking position to create a vacuum in said syringe.

10. The body fluid aspiration and injection syringe of claim 9, wherein said distal end of said longitudinal bore is adapted to be connected to an oxygen source such that oxygen is supplied to the interior of said syringe by passing through said check valve and said holes in said cap seal structure in order to introduce oxygen into blood by foaming oxygen therein.
